(19) United States
(12) Patent Application Publication
(10) Pub. No.: US 2018/0289263 A1
(43) Pub. Date: Oct. 11, 2018

(54) DEVICES AND METHODS FOR ENDOSCOPIC OPTICAL ASSESSMENT OF TISSUE HISTOLOGY

(71) Applicants: Nan M. Jokerst, Durham, NC (US); Deborah A. Fisher, Durham, NC (US); Katherine S. Garman, Durham, NC (US); Benjamin A. Lariviere, Durham, NC (US)

(72) Inventors: Nan M. Jokerst, Durham, NC (US); Deborah A. Fisher, Durham, NC (US); Katherine S. Garman, Durham, NC (US); Benjamin A. Lariviere, Durham, NC (US)

(21) Appl. No.: 15/941,948

(22) Filed: Mar. 30, 2018

Related U.S. Application Data
(60) Provisional application No. 62/479,071, filed on Mar. 30, 2017.

(51) Int. Cl. A61B 5/00; A61B 1/05; A61B 1/06; A61B 1/07; A61B 1/31; A61B 10/04; H01L 27/146; H01L 31/0392; G01J 3/42; G01J 3/28; G01J 3/02

(52) U.S. Cl. CPC A61B 5/0075; A61B 1/05; A61B 1/0669; A61B 1/0684; A61B 1/07; A61B 1/31; G01J 3/0218; A61B 5/0084; H01L 27/14643; H01L 27/14607; H01L 31/03926; G01J 3/42; G01J 3/2803; A61B 10/04

(57) ABSTRACT

Systems and methods are described in this disclosure for an endoscopic optical system comprising a diffuse reflectance spectroscopy probe. The probe includes a light source and a plurality of photodetectors. Each photodetector is positioned at a difference distance from the light source such that an interrogation volume of each photodetector is based at least in part on the distance between the photodetector and the light source. In some implementations, each photodetector of the plurality of photodetectors is at least partially annular in shape and the photodetectors of the plurality of photodetectors are arranged concentrically.

### RELATED APPLICATIONS

This application claims the benefit of U.S. Provisional Application No. 62/479,071, filed Mar. 30, 2017, entitled “DEVICES AND METHODS FOR ENDOSCOPIC OPTICAL ASSESSMENT OF TISSUE HISTOLOGY,” the entire contents of which are incorporated herein by reference.

### BACKGROUND

The present invention relates to systems and methods for endoscopic screening and surveillance. Endoscopic screening and surveillance is used, for example, for the detection, identification, and analysis of neoplastic lesions of the colon (e.g. colorectal cancer screening, adenoma surveillance, surveillance of inflammatory bowel disease (IBD) for detection of dysplasia (IBD surveillance)) and of intestinal metaplasia of the esophagus (i.e. Barrett's esophagus). Currently, these endoscopic assessments are performed using white light endoscopy (WLE). During WLE, potential lesions, such as colon polyps, are resected while the current surveillance approach for high-risk tissues (e.g. Barrett's esophagus and long-standing IBD) involves random biopsies taken throughout affected areas.

Limitations of the conventional approach include removing non-neoplastic tissue due to the inability to make a high certainty visual diagnosis, incompletely resecting abnormal (neoplastic, malignant) tissue because the borders of the normal vs abnormal tissue cannot be determined at the time of endoscopy, and missing abnormal tissue, especially flat lesions because they are difficult to detect with WLE. After a polyp is resected, it can be difficult to determine by white light endoscopy if there is residual polyp tissue. Currently, standard of care after a large polyp has been removed in a piecemeal fashion is a repeat endoscopy at three months to assess for recurrence. Even polypoid lesions can be difficult to detect by WLE; a recent study found an overall adenoma miss rate of 22% across 465 colonoscopies (Pasha, Shabana F., et al. “Comparison of the yield and miss rate of narrow band imaging and white light endoscopy in patients undergoing screening or surveillance colonoscopy: a meta-analysis.” The American journal of gastroenterology 107.3 (2012): 363-370). Similarly, finding dysplasia within areas of IBD or Barrett's is based on random sampling strategy and histologic assessment in pathology rather than a real-time assessment of histologic features.

### SUMMARY

The present disclosure provides a novel probe for endoscopic optical tissue spectroscopy and methods of making and using the same. The device as described herein improves detection rates for cancerous and high-risk tissues. Additionally, it will aid in improving the diagnosis and outcomes of patients by better informing physicians about a patient's health status during and after endoscopy as well as decreasing procedure time for in-vivo tissue inspection over conventional tissue methods such as chromoendoscopy. Also, in some embodiments, the probes described herein provide for a real-time assessment of histological features that would allow a more targeted approach to biopsies.

The probe, according to the present disclosure, can be optimized for a specific tissue sensing application and is compatible with existing commercial endoscopes such that normal uses of the endoscope functions, such as instrument channel functions and video imaging, are not precluded. Indeed, one of the capabilities is to perform custom optimization of the size and shape and number of the Si photodetectors (PDs) in the probe. This custom optimization is enabled through the custom fabrication of the Si PDs, which is in contrast to the prior art, which utilizes commercial optical fibers, which are limited to specific sizes and shapes. Herein is the first discussion of a custom Si diffuse reflectance spectroscopy (DRS) probe that is compatible with standard endoscope instrument channels and specifically optimized for SRDRS measurement of colon dysplasia and cancer. This probe can also be optimized, with different resulting PD shape, size, and number, for other tissues (other tissues have different characteristics which drive the probe PD optimization), as well as multiple light sources. Further, these photodetectors can be formulated such that they are compatible with integration into or onto endoscopes or as endoscope accessories and/or attachments. This enables a range of DRS and spatially resolved DRS (SRDRS) sensor implementations where the light collection waveguide(s), such as typically used optical fibers, are not required.

One aspect of the present disclosure provides a diffuse reflectance spectroscopy (DRS) probe for attaching to, or incorporated therein, an endoscope, the probe comprising, consisting of, or consisting essentially of, one or more light sources for illumination and one or more photodetectors (PDs), the PDs customized in shape and size for light detection so as to optimize the system for a particular target application.

In some embodiments the light source is selected from the group consisting of fiber, LED, OLED, Laser and combinations thereof. In other embodiments the light source is positioned anywhere on the endoscope, on either end of the endoscope, outside of the endoscope, but connected to the endoscope, outside of the endoscope, but optically connected to the endoscope, on a wrapper of the endoscope, or on an attachment to the endoscope. In certain embodiments, the light source is disposable. In another embodiment, the light source is located inside the photodetector array, next to the photodetector array, on top of the photodetector array, or below the photodetector array.

In another embodiment, the photodetector is selected from the group consisting of a photoconductor, a pn junction, a pin junction, and an avalanche photodetector. In yet another embodiment, the photodetector comprises a material selected from the group consisting of Si, Ge, compound semiconductor, and organic materials. In some embodiments, the photodetector comprises a thickness of about 250 to about 650 microns. In other embodiments, the photodetector comprises a flexible, thin film that may be submicron thick to many microns thick. In some embodiments, the flexible thin film is integrated onto a rigid substrate, integrated onto a flexible substrate, or integrated by means selected from the group consisting of bonding, adhesives and printing. In another embodiment the photodetector is rigid on a flat surface. In some embodiment the flat surface comprises the tip of an optical fiber. In another embodiment the photodetector is rigid on a curved surface. In some embodiments the photodetector is folded or curled on a flexible substrate. In another embodiment the photodetector is on a stretchable substrate.

In yet another embodiment, the probe further comprises, consists of, or consists essentially of an optical, electrical, or electromagnetic component. In certain embodiments the optical component comprises a lens. In other embodiments, the probe is optimized, with different resulting PD shape, size, and number, for other tissues (other tissues have different characteristics which drive the probe PD optimization) in a small probe format.

Another aspect of the present disclosure provides a method of using the probe as described herein for non-invasively conducting an “optical biopsy” over areas that are much larger than areas that are currently randomly biopsied. Another aspect of the present disclosure provides a method of using a probe as described herein for characterizing tissue as a function of depth, thereby being able to detect polyps behind folds, which would otherwise be missed in typical endoscopic procedures, using a probe as described herein. Another aspect of the present disclosure provides a method of using a probe as described herein for identifying the tissue margin on large polyps and lesion before removal to guide the resection and removal of the entire lesion so that further resection is not needed.

In one embodiment, the invention provides an endoscopic optical system comprising a diffuse reflectance spectroscopy probe. The probe includes a light source and a plurality of photodetectors. Each photodetector of the plurality of photodetectors is at least partially annular in shape and the photodetectors of the plurality of photodetectors are arranged concentrically. Each photodetector is positioned at a difference distance from the light sources such that an interrogation volume of each photodetector is based at least in part on the distance between the photodetector and the light source.

Other aspects of the invention will become apparent by consideration of the detailed description and accompanying drawings.

### BRIEF DESCRIPTION OF THE DRAWINGS

FIG. 1 is a perspective view of a multispectral endoscopic optical sensor (MEOS) integrated into the tip of an optical fiber in accordance with one embodiment.

FIG. 2A is a front view of a distal end of an optical sensor similar to the optical sensor of FIG. 1.

FIG. 2B is a cross-sectional view of the optical sensor of FIG. 2A.

FIG. 3 is a perspective view of a distal end of an endoscope.

FIG. 4 is a perspective view of a distal end of an endoscope with the optical sensor of FIG. 1 incorporated therein.

FIG. 5 is a perspective view of a distal end of an endoscope with the optical sensor of FIG. 1 wrapped around the exterior of the endoscope.

FIG. 6 is a perspective view of a distal end of an endoscope with a support arm extending from the distal end supporting an optical sensor and/or a light source.

FIG. 7A is a perspective view of a device for retrofitting an optical sensor or a light source to an endoscope.

FIG. 7B is a perspective view of the device of FIG. 7A coupled to the endoscope of FIG. 3.

FIG. 8 is a schematic diagram of an external light source coupled to the optical sensor of FIG. 1 by a waveguide.

FIG. 9 is a perspective view of a multispectral endoscopic optical sensor (MEOS) in accordance with another embodiment.

FIGS. 10A and 10B are perspective views of the optical sensor of FIG. 9 coupled to an endoscope according to one embodiment.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

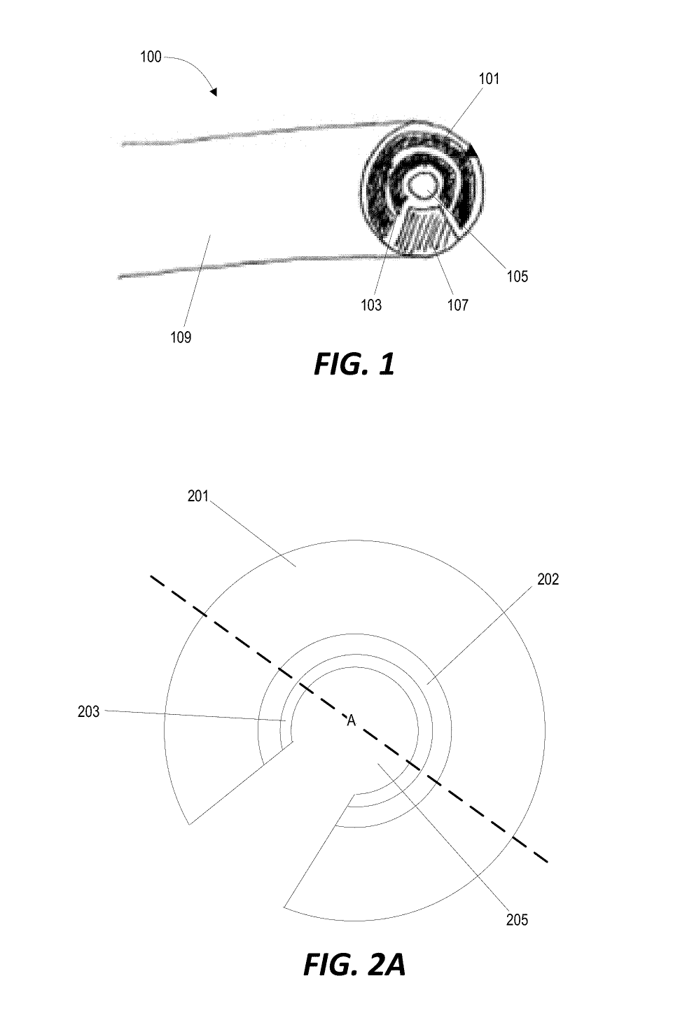

FIG. 1 illustrates an example of an optical probe 100 for spatially-resolved diffuse reflectance spectroscopy (SRDRS). The probe 100 includes a plurality of concentric, partially-annular, thin-film silicon (Si) photodiodes (PDs) 101, 103 for photon collection with a via 107 and optical fiber 109 for light delivery. Light is conveyed by the optical fiber 109 to the probe at its distal end where is it then emitted through an aperture 105 positioned at the center of the partially-annular photodiodes 101, 103. In the example of FIG. 1, the probe 100 includes two photodiodes 101, 103, each positioned with a different thickness at a different distance from the aperture 105.

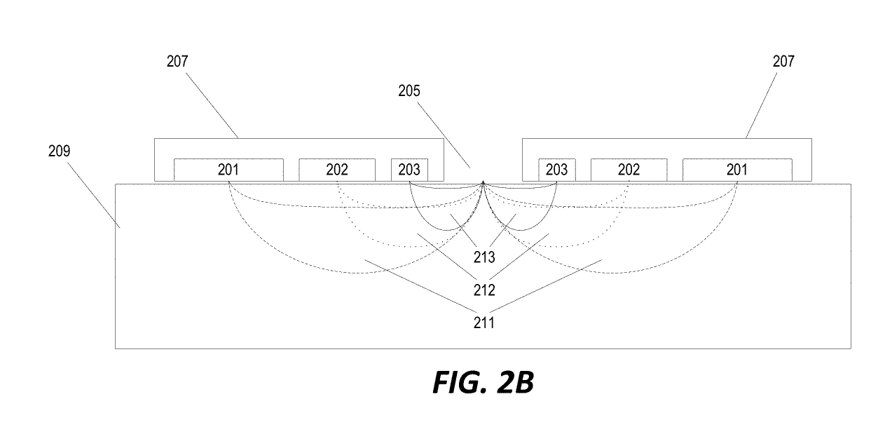

FIGS. 2A and 2B illustrate another example of an optical probe similar to the optical probe 100 of FIG. 1. FIG. 2A illustrates a front (i.e., tissue facing) side of the optical sensor. The optical sensor of FIG. 2A includes three concentric, partially-annular, thin-film Si photodiodes 201, 202, 203. The photodiodes 201, 202, 203 are sized to have different widths and to be positioned at different distances from an aperture 205 positioned at the center of the concentric photodiodes 201, 202, 203. FIG. 2B shows a cross-sectional view of the same optical probe along line “A” in FIG. 2A.

As illustrated in FIG. 2B, each photodiode 201, 202, 203 is bonded to a 500 μm thick transparent glass substrate 207. The photodiodes 201, 202, 203 in this example are fabricated beginning with P-type silicon-on-insulator (SOI) with a 10 μm thick Si device layer on a 1 μm buried oxide. After n-type junctions were patterned and diffused from a spin-on phosphorus source, the sensor array was patterned using reactive ion etching and transferred and metal/metal bonded to the glass substrate.

When the PD array (including photodiodes 201, 202, 203) is placed in direct contact with the tissue 209 to be measured, light is delivered through the aperture 205 of the innermost photodiode 203. The size (e.g., width) and position of each photodiode 201, 202, 203 relative to the aperture 205 provide different interrogation volumes for each of the three photodiodes 201, 202, 203. As illustrated in FIG. 2B, the outermost photodiode 201 in this example provides the largest interrogation volume 211 while the middle photodiode 202 provides a smaller interrogation volume 212 and the innermost photodiode 203 provides the smallest interrogation volume 213. FIG. 2B also illustrates that the depth of the interrogation volume is influenced by the size and position of the interrogation volume relative to the light source/aperture 205. Accordingly, the outermost photodiode 201 captures diffuse reflectance data from greater depths within the tissue 209 while the diffuse reflectance data captured by the innermost photodiode 203 is from nearer to the surface of the tissue 209.

In various different implementations, the size, shape, and orientation of the photodiodes (e.g., photodiodes 201, 202, 203) and their relationship to an optical light source (e.g., light from the aperture 205) can be adjusted and customized for collection of diffuse reflectance spectroscopy (DRS) signals that are optimized for a specific sensing tissue target or application (or sets of tissues or applications). For example, to optimize the design for SRDRS measurement of colon dysplasia, reported scattering and absorption coefficients for normal and adenomatous colon polyps can be used as parameters in a 3D Monte Carlo (MC) model to optimize the probe detector dimensions within conventional endoscope geometrical constraints. The tissue optical properties used in one example of this type of modeling are provided in Table 1 below.

TABLE 1

| | |
|---|---|
| Adenomatous | Normal |
| (mm⁻¹) | (mm⁻¹) |

| λ (nm) | μₐ | μₛ | μₐ | μₛ |
|---|---|---|---|---|
| 415 | 4.7 | 11 | 2.6 | 13 |
| 430 | 3.6 | 9.2 | 1.4 | 11 |
| 515 | 0.62 | 5.5 | 0.2 | 7.6 |
| 540 | 1.1 | 5.5 | 0.36 | 7.3 |
| 555 | 0.86 | 5.4 | 0.24 | 6.9 |
| 577 | 1 | 5.3 | 0.33 | 6.6 |
| 650 | 0.09 | 4.1 | 0.04 | 5.3 |

MC modeling was used to determine the wavelength-dependent reflectances for the 3 PDs using the wavelengths in Table 1, which were then used to optimize the geometry of the PDs. First, for spatial resolution, the Si PD area was divided into three pixels (i.e., the three photodiodes 201, 202, 203). Next, the geometry of the three PDs was optimized to satisfy the following three design criteria: (1) maximizing collected optical signal, (2) signal-to-noise ratio for each PD greater than 40 dB, and (3) to maximize a contrast figure of merit. IN all cases, semi-continuous radially dependent reflectance distributions obtained through MC simulation were integrated to represent different PD geometries.

Optimization of criteria (1) sought to balance the three PD outputs so that no single PD signal was larger than any other (and no single PD was limited by low photocurrent). The highest absorption wavelength (415 nm) was used to assess the PD photocurrents. As the size of the innermost photodiode 203 increases, the photocurrent from the other two photodiodes 202, 201 decreases due to less available width and further distance from the aperture 205. The three photodiodes 201, 202, 203 were balanced in this example at a maximum photocurrent of 86 pA when the width of the innermost photodiode 203 was 58 μm.

Optimization of criteria (2) sought signal-to-noise ratio greater than 40 dB for each of the three photodiodes 201, 202, 203 across all wavelengths. To satisfy these criteria, the DRS photocurrent was simulated for all combinations of photodiode widths between 50 μm and 150 μm for the middle photodiode 202 and widths between 200 μm and 800 μm for the outermost photodiode 201 (using a 2 μm step size). The signal-to-noise ratio was computed for all three photodiodes 201, 202, 203 across all modeled wavelengths. The signal-to-noise ratio decreases as a function of increasing width for both the middle photodiode 202 and the outermost photodiode 201, which is due to the increased contribution of dark current for larger photodiodes.

Optimization of criteria (3) sought to maximize a contrast (Γ) figure of merit. The contrast, defined by the equation:

$$Γ ( λ , {PD}_{i} ) = ( 1 - \frac{{I}_{{signal}_{adenomatous} ( λ , {PD}_{i} )}}{{I}_{{signal}_{normal} ( λ , {PD}_{i} )}} ) \qquad ( 1 )$$

was calculated for each wavelength for each of the three photodiodes 201, 202, 203. The mean of these contrasts was calculated for each photodiode and the figure of merit (FOM) is the product of these three mean contrasts. This FOM is then plotted as a colored contour function of the photodiode widths.

Based on this analysis, all three criteria were satisfied for interrogation of the human colon mucosa for SRDRS measurement of colon dysplasia by photodiode widths of 58 μm, 100 μm, and 550 μm for the innermost photodiode 203, the middle photodiode 202, and the outermost photodiode 201, respectively. Using this determined device geometry, SE-SRDRS sensors were fabricated using standard microfabrication processing techniques beginning with 4-inch P-type silicon-on-insulator material. Briefly, after PN junctions were diffused in the 10 μm thick SOI device layer, the device layer was patterned into a semi-annular mesa and transferred to a transparent Pyrex substrate. Finally the device was integrated with a printed circuit board and mounted to couple with an optical fiber.

The same probe and photodiode array illustrated in FIGS. 2A and 2B can also be optimized for other specific uses and application, for example, interrogation and assessment of the esophagus, by adjusting the photodiode geometries (e.g., the widths of each of the concentric partially-annular photodiodes). Other implementations can also be adjusted to include more or fewer photodiodes.

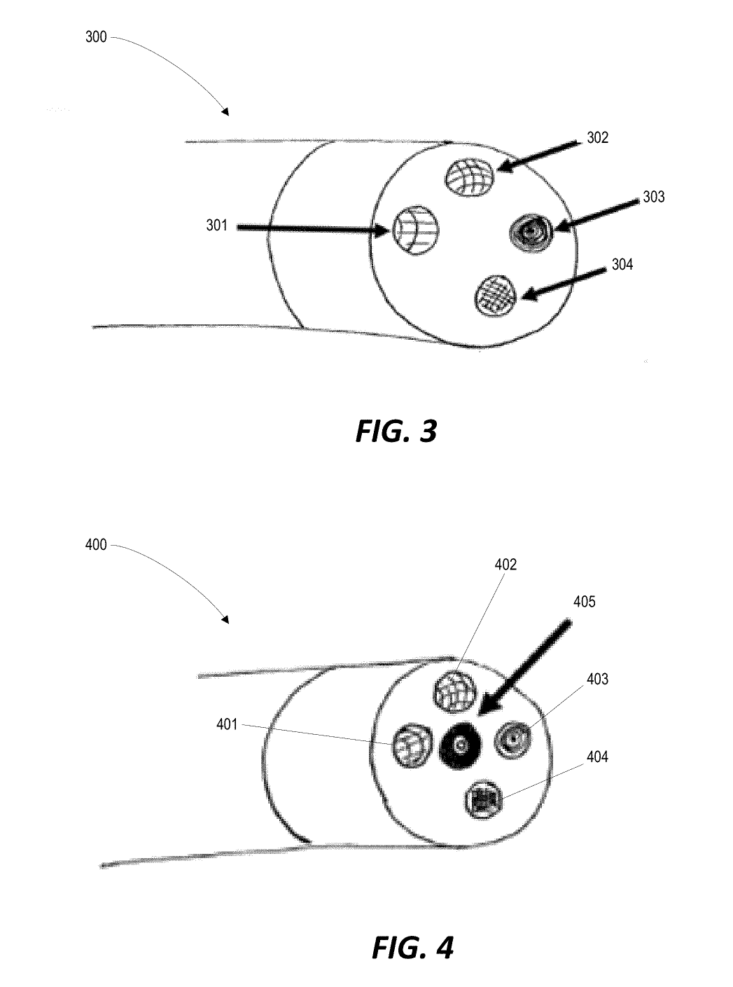

The optical probes such as discussed above in reference to the examples of FIGS. 1, 2A, and 2B can be adapted for use with an endoscope. FIG. 3 illustrates an example of an endoscope 300 with an instrument/biopsy channel 301 opening at the distal end of the endoscope 300. A light illumination source channel 302, an air/water nozzle 303, and an objective 304 for video imaging are also positioned at the distal end of the endoscope 300. In some implementations, an optical probe, such as optical probe 100 illustrated in FIG. 1, can be utilized with the endoscope 300 of FIG. 3 by extending the optical probe through the instrument/biopsy channel 301 of the endoscope 300. However, in other implementations, the optical probe can be integrated and utilized with an endoscope in other configurations.

FIG. 4 illustrates another example of an endoscope 400. Like the example of FIG. 3, this endoscope 400 also includes an instrument/biopsy channel 401, a light illumination source channel 402, an air/water nozzle 403, and an objective 404 for video imaging. However, the optical sensor/probe 405 such as illustrated in FIGS. 1, 2A, and 2B is also integrated into the endoscope 400 and positioned at the distal end of the endoscope 400. In implementations where the optical sensor 405 is coupled to the distal end of an optical fiber (as in the example of FIG. 1), the endoscope 400 might be configured to house the optical fiber within the body of the endoscope 400.

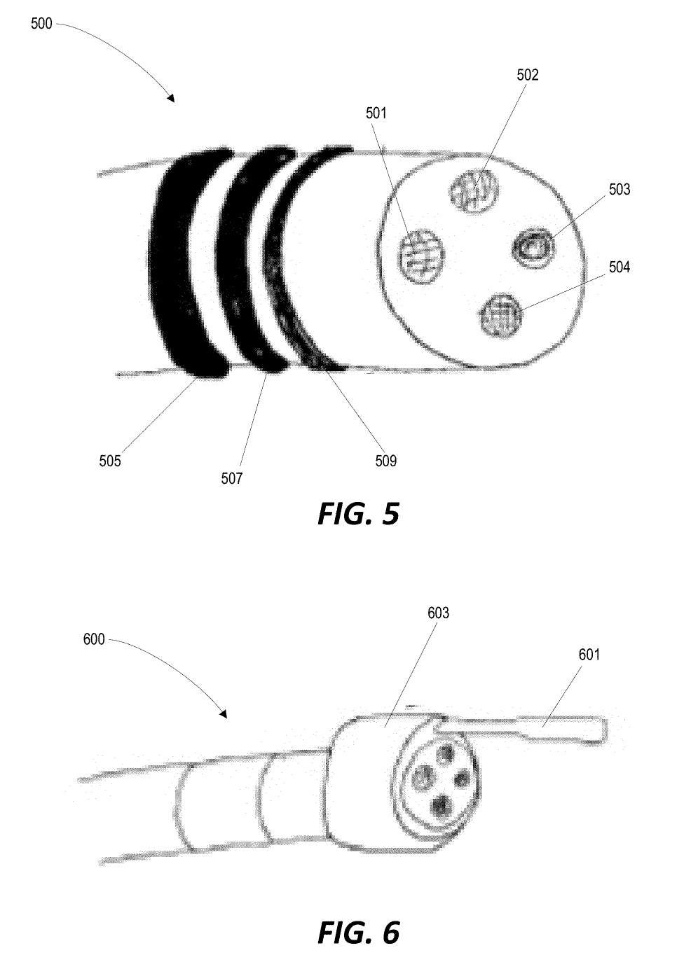

FIG. 5 illustrates another example of an endoscope 500 that includes an instrument/biopsy channel 501, a light illumination source channel 502, an air/water nozzle 503, and an objective 504 for video imaging. However, in this example, instead of integrating the optical sensor into the distal end of the endoscope, the optical sensor is wrapped around the exterior of the endoscope housing near the distal end. In this particular, example, a photodiode array including two photodiodes 505, 507 are positioned concentrically around the body of the endoscope 500. Similarly, an illumination aperture 509 for providing light utilized by the photodiodes 505, 507 is also positioned around the endoscope 500 near its distal end. In this example, the geometry of the photodiode array can be adjusted, for example, by adjusting the width of each photodiode in a linear direction parallel to the length of the endoscope 500. Alternatively, the geometry of each photodiode can be adjusted by increasing or decreasing the degree to which the photodiode extends radially from the endoscope 500. Similarly, the position of each photodiode from the illumination aperture 509 can be varied by changing the linear position of the photodiode along the length of the endoscope 500 or by changing the radial position of the photodiode (i.e., how far the photodiode extends from the endoscope surface).

Similar to the examples discussed above, in the implementation of FIG. 5, each photodiode 505, 507 and the illumination aperture 509 can be coupled to one or more optic fibers and/or waveguides positioned within the endoscope 500. Alternatively, instead of utilizing optic fibers that run the length of the endoscope 500, in some implementations, the photodiodes 505, 507 can be coupled to an electronic processor positioned in the endoscope 500 near its distal end. Similarly, in some implementations, the illumination aperture 509 may be coupled to a light source positioned within the endoscope 500 near its distal end.

FIG. 6 illustrates an example of an endoscope 600 in which a support arm 601 extends from a collar 603 positioned around the distal end of the endoscope 600. In this example, the photodiode array and/or the light source may be positioned on the support arm 601. In the example of FIG. 6, the optical probe positioned on the support arm 601 may include an array of photodiodes and an illumination aperture coupled to one or more optic fibers and/or waveguides that are housed within the endoscope 600. For example, the illumination aperture positioned on the support arm 601 may be coupled to an external light source by an optic fiber and/or waveguide. Alternatively, the endoscope 600 may be configured to include a light source positioned near the distal end of the endoscope 600 or even within the support arm 601. Similarly, the photodiodes of the optical probe positioned on the support arm 601 may be coupled by one or more optic fibers and/or waveguides to an external electronic processor or, alternatively, may be coupled to an electronic processor positioned within the endoscope 600 or even within the support arm 601.

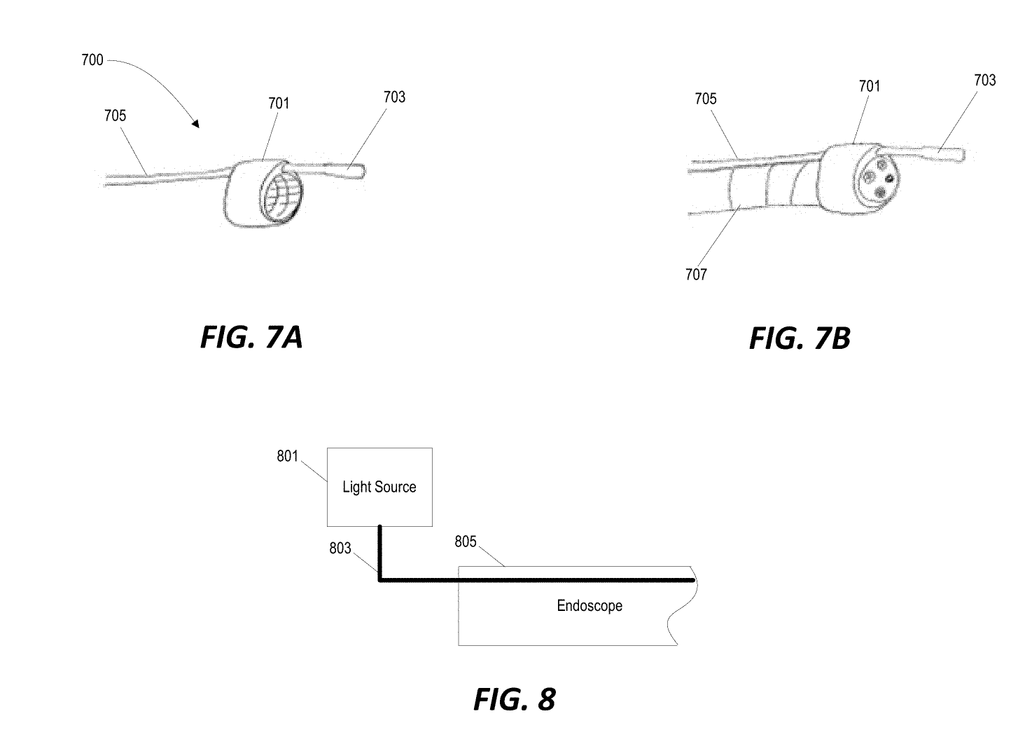

FIGS. 7A and 7B illustrates a similar configuration used to retrofit an endoscope for use with a new optical probe (such as illustrated in the examples of FIG. 1 and FIGS. 2A and 2B above). As shown in FIG. 7A, a device 700 includes a collar 701 and a support arm 703 extending from the collar 701. In the example of FIG. 7A, one or more cables, optical fibers, and/or waveguides 705 extend from the collar 701 to an external system. As illustrated in FIG. 7B, the device 700 of FIG. 7A is coupled to an endoscope 707 by placing the collar 701 around the distal end of the endoscope 707. The one or more cables, optical fibers, and/or waveguides 705 are then positioned to extend from the collar 701 along the length of the endoscope 707.

In any of the example described herein, the light source may include any light source that is capable of illuminating a tissue, and the system may contain one or more light sources. The light sources may be standard thickness or thin film. Examples of types of light sources include, but are not limited to fiber, white light source, LED, OLED, Laser, and combinations thereof. Furthermore, in various different implementations, the light source may be positioned on the endoscope, inside the endoscope, outside the endoscope, and at either end of the endoscope. For example, as discussed above, in some implementations of the system illustrated in FIG. 6, a light source is provided on the support arm 601 extending from the color 603 of the endoscope 600. However, in other implementations, as illustrated in FIG. 8, a light source 801 is positioned external to the proximal end of the endoscope 805 and is coupled to the optical probe by an optic fiber and/or waveguide 803 that extends through the length of the endoscope 805.

In various implementations, the optical probe and/or the endoscope may be configured to such that the light source is positioned outside the endoscope but connected to the endoscope (e.g., as in the example of FIG. 6) or outside of the endoscope, but optically connected to the endoscope (e.g., as in the example of FIG. 8). In other implementations, the light source may be on a wrapper of the endoscope (e.g., as in the example of FIG. 4) or on an attachment to the endoscope (e.g., as in the example of FIG. 7B). In some implementations, the light source is disposable and, in some implementations, the light source is located inside the photodiode array (e.g., next to the photodiode array, on top of the photodiode array, or below the photodiode array).

Furthermore, although the examples presented above describe examples of optic probes that utilize an array of photodiodes, in some implementations, other types of photodetectors may be utilized. For example, the photodetectors may include any device that is capable of sensing light or other electromagnetic energy. Examples include, but are not limited to, a photoconductor, a pn junction, a pin junction, an avalanche photodetector and the like. The material of the photodetector/photodiode may include, for example, Si, Ge, compound semiconductors, organic materials, and the like. The thickness and geometry of the photodetector (and the photodetector array) may be varied to allow for its incorporation into a particular device (e.g., an endoscope) and to customize the imaging component for a particular use and/or a particular tissue type.

In the examples discussed above, the photodetector array is provided as a series of concentric, partially-annular, flexible thin-film photodiodes. In some implementations, the flexible thin film is integrated onto a rigid substrate or onto a flexible or stretchable substrate. In some implementations, the thin-film photodiodes are integrated onto the substrate by bonding, adhesives and/or printing techniques. The photodetectors in the array can be implemented in a variety of different shapes such as, for example, a “donut” or an arc. Moreover, in various different implementations, the photodetectors can be of the same shape and/or size or of different shapes and/or sizes. In some implementations, as discussed above, the geometry of the photodetectors is optimized for a specific target tissue application or a set of multiple tissues/applications.

The light sources may also be present in either single or array format, and in thick or thin film, and in any shape or orientation with respect to the photodetector or photodetectors. In some implementations, a flexible thin film light source is integrated onto a rigid substrate or onto a flexible or stretchable substrate. In some implementations, thin-film light sources are integrated onto the substrate by bonding, adhesives and/or printing techniques. The light sources can be implemented in a variety of different shapes such as, for example, a series of dots or lines or an arc. Moreover, in various different implementations, the light sources can be of the same shape and/or size or of different shapes and/or sizes, and may interleave with the photodetectors. In some implementations, as discussed above, the geometry of the light sources is optimized for a specific target tissue application or a set of multiple tissues/applications.

In the examples of FIG. 1 and FIGS. 2A and 2B, the photodetector is provided as a rigid structure on a flat surface (e.g., a glass substrate and/or the tip of an optical fiber). However, in other implementations, the photodetector(s) and/or light source(s) may be implemented on a curved surface and/or on a flexible surface. In some implementations, the photodetector(s) and/or light source(s) may be implemented on a flexible and/or stretchable substrate. FIG. 9 illustrates an example of an optical probe 900 with a curved substrate 901. Two photodiodes 903, 905 and a light source 907 are formed on the curved substrate 901. In this example, the curved substrate 901 may be rigid or may be flexible. In implementations where the substrate 901 is a flexible material, the photodetector may be formed on a substrate as illustrated in FIG. 9 and then curled or rolled in order to position the photodiodes in a concentric, partially-annular arrangement.

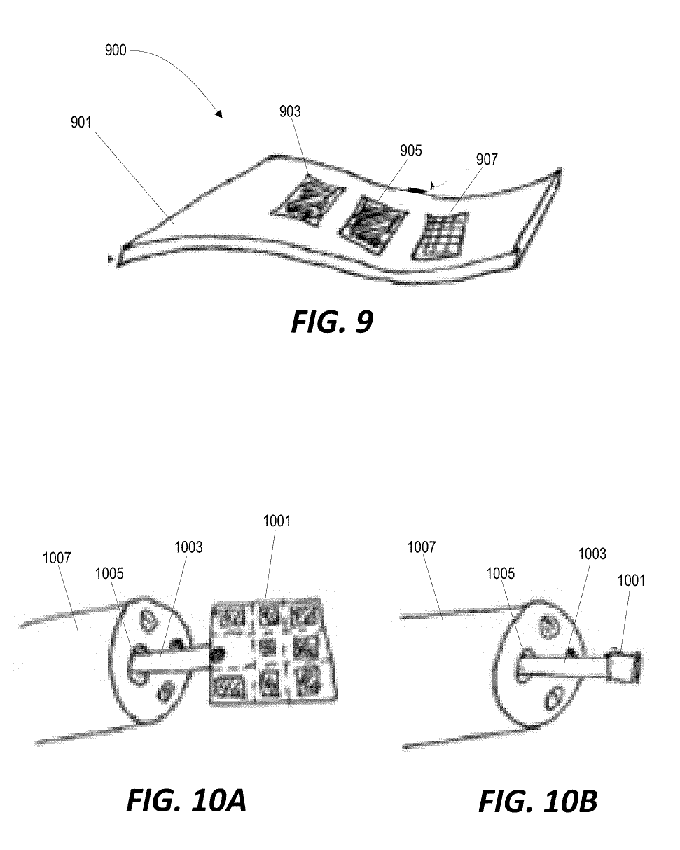

FIGS. 10A and 10B illustrate an example in which a photodetector array 1001 is formed by integrating an array of photodiodes onto a surface of a flexible substrate. The photodetector array 1001 is then rolled at the end of an optic fiber 1003 which is positioned within an instrument channel 1005 of an endoscope 1007.

In some implementations, the optic probe may include additional optical, electrical, or electromagnetic components. For example, the optic probe may be configured to include a lens and/or an electronic processor to detect and quantify light received by the photodetector array, and/or to amplify or digitize signals from the photodetector, or to drive and/or shape the pulse of the light source, which may be continuously emitting or have output light intensity shaped in time. Furthermore, as shown in various different examples above, an optical probe in accordance with this invention may be implemented, for example, inside an endoscope (e.g., in a channel, such as the instrument channel in the example of FIGS. 10A and 10B), on the outside of the endoscope as a flexible disposable “wrapper” (e.g., arranged as in the example of FIG. 5), as an accessory that can be disposable (e.g., as in the example of FIGS. 7A and 7B), or permanently integrated with the endoscope and can be cleaned/reused with the endoscope (e.g., arranged as in the example of FIG. 4).

The DRS probe, such as described in the various examples above, can be used to non-invasively conduct an “optical biopsy” over areas that are much larger than areas that might otherwise be randomly biopsied. This is particularly important for surveillance for dysplasia in the setting of long-standing IBD (ulcerative colitis, Crohn's disease)—as an alternative to using dye for chromoendoscopy or to obtaining random biopsies. While random biopsies demonstrate low sensitivity to detect dysplasia due to the very small percentage of the total area sampled, “optical biopsies” using the systems described herein can be performed on the entire colon or esophagus much faster than would be possible with other optical techniques, and much more accurately than current endoscopic tissue sampling protocols.

In some implementations, data captured by the DRS sensor can be used, for example, to characterize tissue as a function of depth, and thus are able to detect polyps behind folds, which would otherwise be missed in typical endoscopic procedures. While other endoscopic techniques to find lesions behind a colonic fold/within a flexure are limited to using the scope itself or a cap at the end of the scope to help flatten the fold, the DRS sensor described herein allows detection of an abnormality through the fold. There is no other in vivo endoscopic technology to accomplish this task.

The DRS sensor can characterize flat polyps, and the margins of flat polyps, which are often missed entirely or which are incompletely removed using current endoscopic biopsy techniques. A missed polyp or incompletely removed polyp can lead to an avoidable cancer or recurrent adenoma. The only current alternative to incompletely removed polyps is to mark the suspected lesion with sterile India ink (i.e. a tattoo), then repeat the colonoscopy at approximately 2-4 months to endoscopically observe and biopsy the area. This leads to a longer procedure time for the initial colonoscopy (e.g. time to tattoo areas adjacent to suspected lesions) and additional colonoscopies that would otherwise not be required. To avoid recurrence, colonoscopies are repeated every 2-4 months until no abnormal tissue is detected, which frequently results in the need for a single patient to undergo numerous colonoscopies in the span of 1 to 2 years. In contrast, the systems and methods described herein provide physicians with a higher rate of detection for high risk tissue during an initial procedure, which in-turn increases the physicians' confidence in recommending a longer period of time between subsequent follow-up procedures.

The DRS sensors described herein can also reduce the time of colonoscopy for IBD surveillance using conventional techniques such as spray dye chromoendoscopy. Many endoscopy units schedule colonoscopy for IBD surveillance as two procedure slots. Use of the DRS sensor could reduce that time to the expected time for routine endoscopy. In addition, the low sensitivity of conventional IBD surveillance has led to recommendations for frequent colonoscopy (every 1-2 years). A more accurate technique, such as provided by using the DRS sensors described herein, could increase this interval and thereby reduce the number of colonoscopies over a patient's lifetime.

Data captured by the DRS sensor can be used to identify the tissue margin on large polyps and lesion before removal to guide the resection and removal of the entire lesion so that further resection is not needed. This enhances, for example, the effectiveness and safety of large polyp removal via endoscopic mucosal resection (EMR) and endoscopic submucosal dissection (ESD) in the upper and lower GI tract through in vivo histologic assessment of the lesion's depth and borders. There are no other techniques to assess for complete resection of these large, flat lesions. The current technique is to tattoo adjacent to the lesion and repeat the colonoscopy with biopsy in 2-4 months. Use of the DRS sensor will both reduce the time of the initial colonoscopy by providing clear information of complete resection (i.e. negative margins) it could obviate the need (and therefore procedure time) to mark the area with a tattoo. The DRS sensor can also be used to identify the tissue margin on large polyps and lesions after removal and resection of the lesion to identify if further resection is not needed.

Thus, the invention provides, among other things, an endoscopic optical system for diffuse reflectance spectroscopy including an optical probe with an array of spatially-resolved photodetectors. Various features and advantages are set forth in the following claims.

What is claimed is:

1. A endoscopic optical system comprising a diffuse reflectance spectroscopy probe including:
a light source; and
a plurality of photodetectors, wherein each photodetector of the plurality of photodetectors is positioned at a different distance from the light source or sources such that an interrogation volume of each photodetector is based at least in part on the distance between the photodetector and the light source or sources.

2. The endoscopic optical system of claim 1, wherein each photodetector of the plurality of photodetectors is of a similar shape arranged in an array.

3. The endoscopic optical system of claim 2, wherein the light source of the diffuse reflectance spectroscopy probe includes an illumination aperture or light source positioned at the center of the array of similarly shaped photodetectors.

4. The endoscopic optical system of claim 1, wherein each photodetector of the plurality of photodetectors is at least partially annular in shape, wherein the photodetectors of the plurality of photodetectors are arranged concentrically.

5. The endoscopic optical system of claim 4, wherein the light source of the diffuse reflectance spectroscopy probe includes an illumination aperture or light source positioned at the center of the concentrically arranged photodetectors.

6. The endoscopic optical system of claim 5, wherein the illumination aperture is coupled by an optical fiber to at least one selected from a group consisting of a lamp, an OLED, a LED, and a laser.

7. The endoscopic optical system of claim 4, wherein the plurality of photodetectors are arranged concentrically on a distal end of an optic fiber.

8. The endoscopic optical system of claim 4, wherein the plurality of photodetectors are arranged concentrically on a substrate surface, wherein each photodetector of the plurality of photodetectors has a surface area defined by a radial width of the photodetector.

9. The endoscopic optical system of claim 8, wherein a surface area of an innermost photodetector of the plurality of photodetectors is smaller than a surface area of every other photodetector of the plurality of photodetectors.

10. The endoscopic optical system of claim 8, wherein a surface area of an outermost photodetector of the plurality of photodetectors is larger than a surface area of every other photodetector of the plurality of photodetectors.

11. The endoscopic optical system of claim 1, wherein the plurality of photodetectors includes a first partially annular photodiode, a second partially annular photodiode, and a third partially annular photodiode arranged concentrically on a glass substrate,
wherein the first partially annular photodiode is positioned nearest to a center of the concentrically arranged plurality of photodetectors and has a surface area defined by a first radial width,
wherein the second partially annular photodiode is positioned adjacent to the first partially annular photodiode and has a surface area defined by a second radial width, wherein the second radial width is larger than the first radial width, and
wherein the third partially annular photodiode is positioned adjacent to the second partially annular photodiode opposite the first partially annular photodiode and has a surface area defined by a third radial width, wherein the third radial width is larger than the second radial width.

12. The endoscopic optical system of claim 11, wherein the diffuse reflectance spectroscopy probe is configured to perform an optical biopsy of human colon mucosa, wherein the first radial width is approximately 58 μm, wherein the second radial width is approximately 100 μm, and wherein the third radial width is approximately 550 μm.

13. The endoscopic optical system of claim 11, wherein the plurality of photodetectors include photodiodes formed of at least one selected from a group consisting of Si, Ge, a compound semiconductor material, and an organic material.

14. The endoscopic optical system of claim 1, wherein each photodetector of the plurality of photodetectors include at least one selected from group consisting of a photoconductor, a pn junction, pin junction, and an avalanche photodiode.

15. The endoscopic optical system of claim 1, wherein the plurality of photodetectors are arranged such that the interrogation volume of each photodetector extends to a different tissue depth when the diffuse reflectance spectroscopy probe is placed against a tissue surface.

16. The endoscopic optical system of claim 1, further comprising an endoscope, wherein the diffuse reflectance spectroscopy probe is integrated into a distal end of the endoscope.

17. The endoscopic optical system of claim 1, further comprising an endoscope with at least one access channel, wherein the diffuse reflectance spectroscopy probe is extended through the at least one access channel to a distal end of the endoscope.

18. The endoscopic optical system of claim 1, further comprising an endoscope, wherein the photodetectors of the plurality of photodetectors are arranged around the endoscope on an exterior body of the endoscope, and wherein each photodetector of the plurality of photodetectors is located at a different position on the endoscope.

19. The endoscopic optical system of claim 1, further comprising an endoscope, wherein the photodetectors of the plurality of photodetectors are arranged concentrically around the endoscope on an exterior body of the endoscope, and wherein each photodetector of the plurality of photodetectors is located at a different linear position along the length of the endoscope.

20. The endoscopic optical system of claim 19, wherein the light source is arranged concentrically around the endoscope on the exterior body of the endoscope.

21. The endoscopic optical system of claim 1, further comprising a support arm extending from a collar positioned around a distal end of an endoscope, wherein the light source is positioned on the support arm.

22. The endoscopic optical system of claim 21, wherein the collar is selectively couplable to the distal end of the endoscope.

23. The endoscopic optical system of claim 21, wherein the light source includes an illumination aperture positioned on the support arm, and further comprising an optical fiber coupling the illumination aperture to an LED or laser light source positioned external to the endoscope through a proximal end of the endoscope.

24. The endoscopic optical system of claim 1, wherein the photodetectors and/or light sources are attached to or printed onto a flexible or stretchable substrate.

25. The endoscopic optical system of claim 1, wherein the photodetectors and/or light sources are thin film devices with a thickness of less than 25 microns.
